(19) United States
(12) Patent Application Publication
(10) Pub. No.: US 2024/0207544 A1
(43) Pub. Date: Jun. 27, 2024

(54) INHALATION FLOW CHAMBER

(71) Applicants: Oscar Ivan Quintero Osorio, Cali (CO); FUNDACIÓN VALLE DE LILI, Cali (CO)

(72) Inventor: Oscar Ivan Quintero Osorio, Cali (CO)

(21) Appl. No.: 18/557,727

(22) PCT Filed: Apr. 28, 2022
(86) PCT No.: PCT/IB2022/053952

§ 371 Date: Oct. 27, 2023

(30) Foreign Application Priority Data
Apr. 28, 2021 (CO) NC2021/0005519

(51) Int. Cl. A61M 15/00; A61M 16/04; A61M 16/06; A61M 16/20

(52) U.S. Cl. CPC A61M 15/0086; A61M 16/049; A61M 16/06; A61M 16/209

(57) ABSTRACT

The invention is related to devices for inhalation therapy that allow optimizing the delivery of the medicament either directly to the mouth, the tracheostomy of the patient, or through the use of an oronasal mask. The inhalation flow chamber (1) comprises a mixed connector (3), an anterior body (19) with an exhalation window (15) housing an exhalation unidirectional membrane (5), an inhalation unidirectional membrane (10), a valve seal (14), a cylinder (18), a coupling port for the pressure indicator (20), a flow indicator (17), a lid for the chamber (7) and a coupling port for the metered-dose inhaler (6).

### FIELD OF THE ART

The present invention is related to medical devices, specifically to devices used in inhalation therapy, which allow optimizing the delivery of the medicament either directly to the mouth, the tracheostomy of the patient, or through the use of an oronasal mask.

### STATE OF THE ART

Inhalation therapy is the administration of medication through the airway of a patient with lung disease. This is performed by a variety of devices that deliver these medications into the airway. Several types of devices are used in inhalation therapy, such as jet nebulizers, ultrasonic nebulizers, dry powder inhalers, and metered-dose inhalers (MDIs).

The MDI is a small, portable device that has a pressurized cartridge which releases the drug when it is pressed. Direct use of the MDI on the patient's mouth is not very recommended, because it requires an adequate inspiration/activation coordination and also causes a high oropharyngeal impact. Due to the above, several devices have been developed that seek to optimize drug delivery, as described below.

Document U.S. Pat. No. 6,578,571B1 relates to a paediatric inhalation device and method including an auxiliary device having an inlet, an outlet, and a chamber. A delivery device attached to the outlet and an inflatable mask of soft and flexible material attached to the delivery device. Likewise, it comprises a T adapter with an inhalation valve and an exhalation valve of approximately the same size visible to the adult observer, a whistle capable of emitting a sound during inhalation.

Patent application US20020104531A1 relates to a paediatric inhalation device and method including an auxiliary device having an inlet, an outlet, and a chamber. A delivery device attached to the outlet and an inflatable mask of soft and flexible material attached to the delivery device. The delivery device comprises a T-adapter with an inhalation valve and an exhalation valve of approximately the same size, which are visible to the adult observer, further comprising a whistle provided in the paediatric inhalation device capable of emitting an audible sound during the inhalation desired by the child.

Patent document U.S. Pat. No. 5,042,467A discloses a medication inhaler including an elongate hollow body for improving medication nebulization. Additionally, it comprises a sound indicator that is exposed to the interior of the inhaler and the outside air. The sound indication device comprises an integrally moulded plastic body and a vibrating tab. If a person inhales too fast, the tab will vibrate by reducing the pressure inside the inhaler, so a pleasant musical tone will be generated to alert the user that they are inhaling too fast.

The sound mechanisms described above allow us to indicate when the increase in inspiratory flow is generated. However, they are not activated if the patient generates very low inspiratory flows, which can favour loss of the medicament by sedimentation. In addition, it does not indicate the ideal inspiratory flow, and does not generate benefits in patients with hearing impairment, cophosis or anacusis.

There are other devices that slow the rate of medication and whose antistatic body keeps the medicament in suspension until it is ready to inhale. However, this mechanism only allows knowing if the patient is performing the inspiration or exhalation and does not indicate the ideal inspiratory flow.

### SUMMARY OF THE INVENTION

In view of the foregoing, there is a need in the technical field for inhalation therapy devices that do not allow loss of medication, that indicate when the ideal inspiratory flow exists, and that can be used in paediatric or tracheostomy patients.

The inventors of the present invention provide a device for optimizing drug delivery by correctly performing the ideal inspiratory flow, favouring inspiration/activation synchronization and educating patients and health care professionals. In addition, it allows administration of the medication directly to the mouth or tracheostomy of the patient, or by using an oronasal mask. Likewise, the position of the flow indicator at the distal end enhances patient comfort, since the patient can see it at an appropriate distance during the intervention.

Throughout the description and claims the word “comprises” and its variants are not intended to exclude other technical features, additives, components or steps. In addition, the word “comprises” includes the case “consists of”. For persons skilled in the art, other objects, advantages and characteristics of the invention will emerge in part from the description and in part from the practice of the invention. The following examples and drawings are provided by way of illustration, and are not intended to be limiting of the present invention. Further, the present invention covers all possible combinations of particular and preferred embodiments indicated herein.

### BRIEF DESCRIPTION OF THE DRAWINGS

FIG. 1 shows a right side view of the inhalation flow chamber, with the metered-dose inhaler and an oronasal mask coupled, in use in a patient without an artificial airway.

FIG. 2 shows a side view of the inhalation flow chamber, with the metered-dose inhaler coupled, in use in a tracheostomy patient.

FIG. 3 shows a side view of the inhalation flow chamber.

FIG. 4 shows a side view of the inhalation flow chamber, and of a metered-dose inhaler and an oronasal mask not coupled to the inhalation chamber.

FIG. 5 shows a side view of the inhalation flow chamber with a coupled metered-dose inhaler.

FIG. 6 shows a side view of the inhalation flow chamber with a metered-dose inhaler and an oronasal mask coupled.

FIG. 7 shows a front view of the inhalation flow chamber.

FIG. 8 shows a front side view of the inhalation flow chamber, where a cross-section of the cylinder, the mixed connector and the anterior body can be observed.

FIG. 9 shows a rear side view of the inhalation flow chamber, where a cross-section of the cylinder, the mixed connector and the anterior body can be observed.

FIG. 10 shows the top view of the inhalation flow chamber.

FIG. 11 shows a left side view of the inhalation flow chamber, with the metered-dose inhaler and an oronasal mask coupled, in use in a patient without artificial airway.

FIG. 12 shows the bottom view of the inhalation flow chamber with a metered-dose inhaler and an oronasal mask coupled.

FIG. 13 shows a rear side view of the inhalation flow chamber.

FIG. 14 shows an exploded side view of the inhalation flow chamber.

FIG. 15 shows a front view of the flow indicator of the inhalation flow chamber.

FIG. 16 shows a diagonal view of the flow indicator of the inhalation flow chamber.

FIG. 17 shows a cross-section of a diagonal view of the flow indicator of the inhalation flow chamber.

FIG. 18 shows an exploded diagonal view of a preferred embodiment of the inhalation flow chamber.

FIG. 19 shows a diagonal view of a preferred embodiment of the inhalation flow chamber with a coupled metered-dose inhaler.

### DETAILED DESCRIPTION OF THE INVENTION

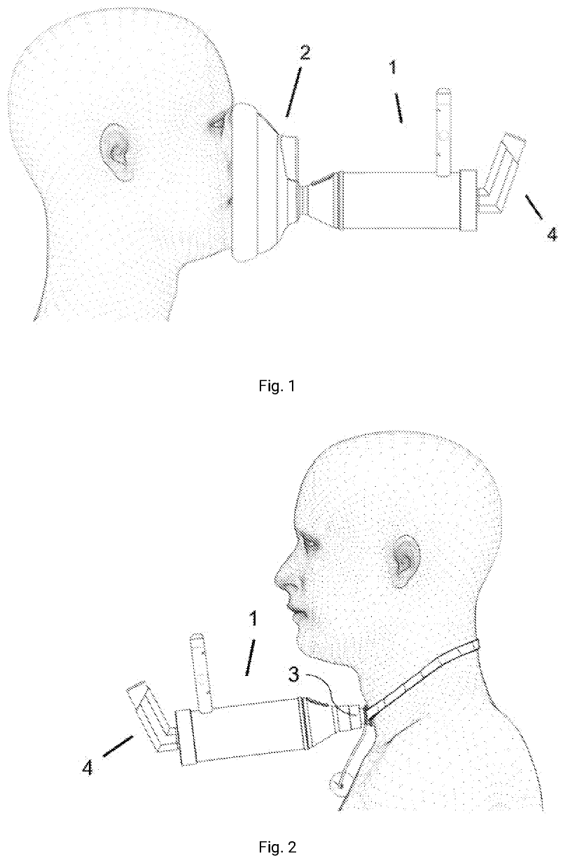

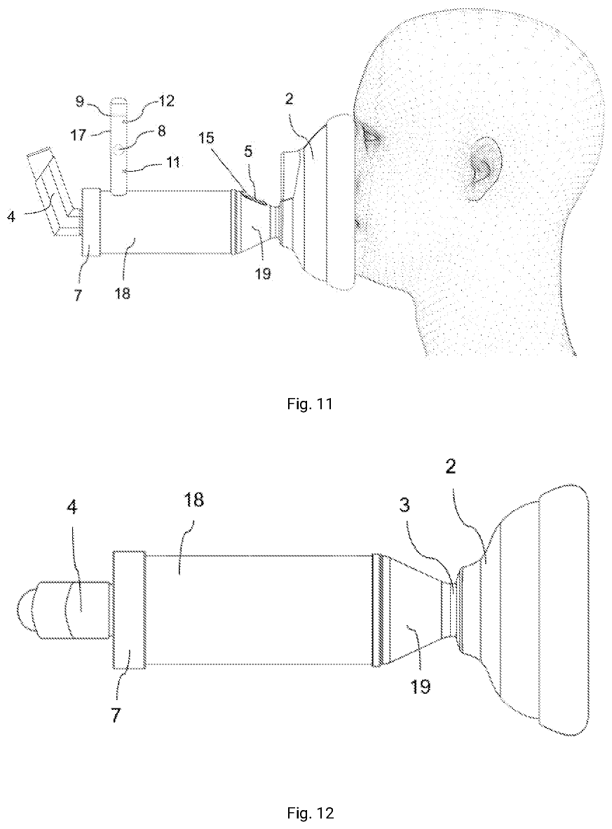

The invention is directed to an inhalation flow chamber (1). The device provided here makes it possible to optimize the delivery of the medication by correctly performing the inspiratory flow, since it has a flow indicator that indicates the ideal levels and favours inspiration/activation synchronization, since the patient can correctly observe the indicator during the intervention and educates the health professionals. It also allows the administration of the medication directly to the mouth or tracheostomy of the patient, as can be seen in FIG. 2, or by using an oronasal mask as can be seen in FIGS. 1 and 11.

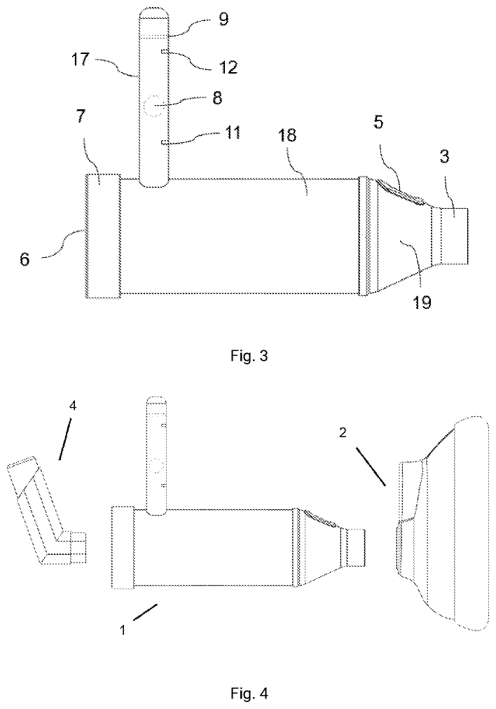

As can be seen in FIGS. 3, 8, 9 and 10, the inhalation flow chamber (1) comprises a mixed connector (3), an anterior body (19) with an exhalation window (15) housing an exhalation unidirectional membrane (5), an inhalation unidirectional membrane (10), a valve seal (14), a cylinder (18), a coupling port for the pressure indicator (20), a flow indicator (17), a lid for the chamber (7) and a coupling port for the metered-dose inhaler (6).

In the proximal section of the device is arranged the mixed connector (3) which is coupled to the previous body (19), which is coupled to the proximal end of the cylinder (18), within which is arranged the inhalation unidirectional membrane (10) and the valve seal (14), which supports said membrane, as shown in FIG. 14.

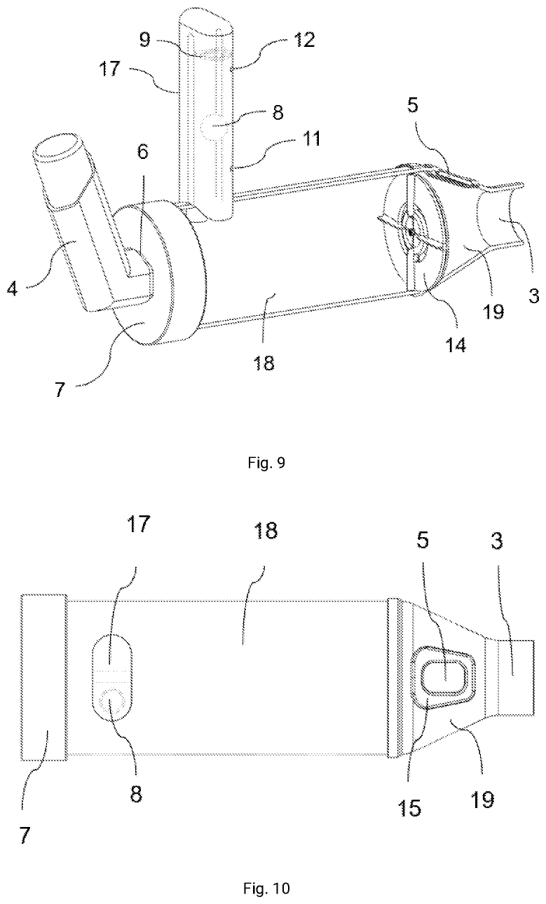

In the central section of the device is the flow indicator (17) that is in connection with the cylinder (18), through the connection of the flow passage conduit (13) with the coupling port (20), as seen in FIGS. 9 and 14.

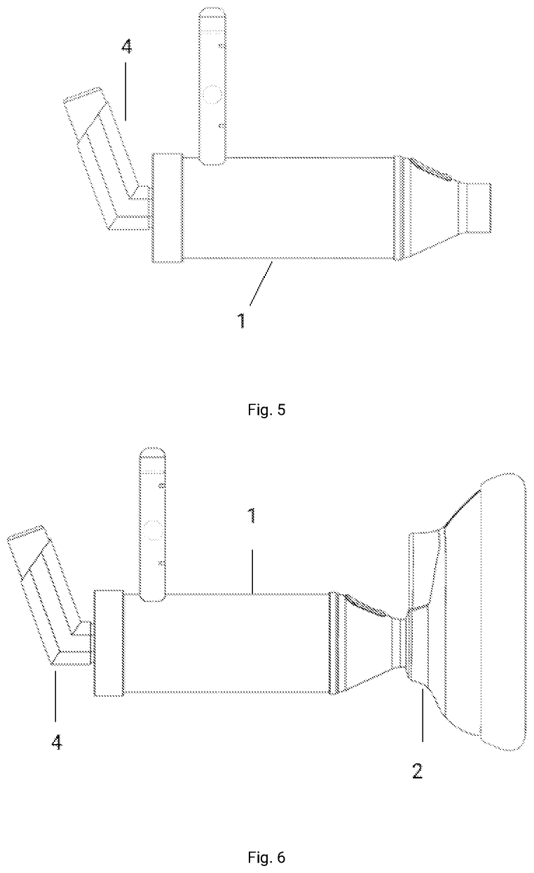

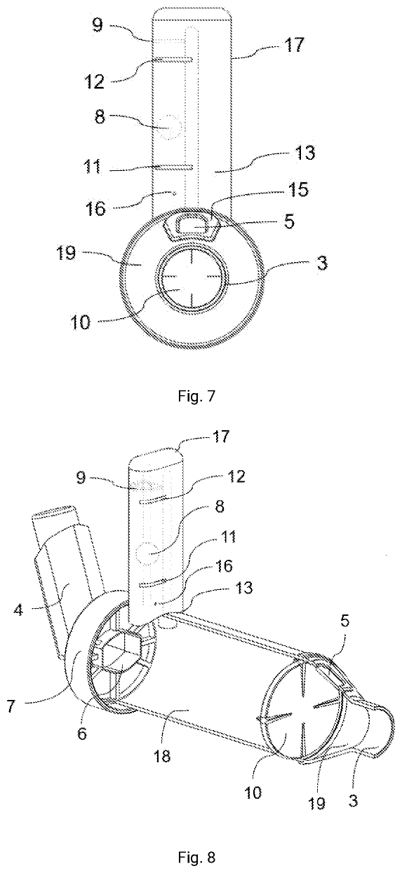

In the distal section of the device is the cylinder (18) in connection with the lid for the chamber (7) with the coupling port (6), to which the metered dose inhaler (4) is coupled, as shown in FIGS. 4, 5, 6 and 12.

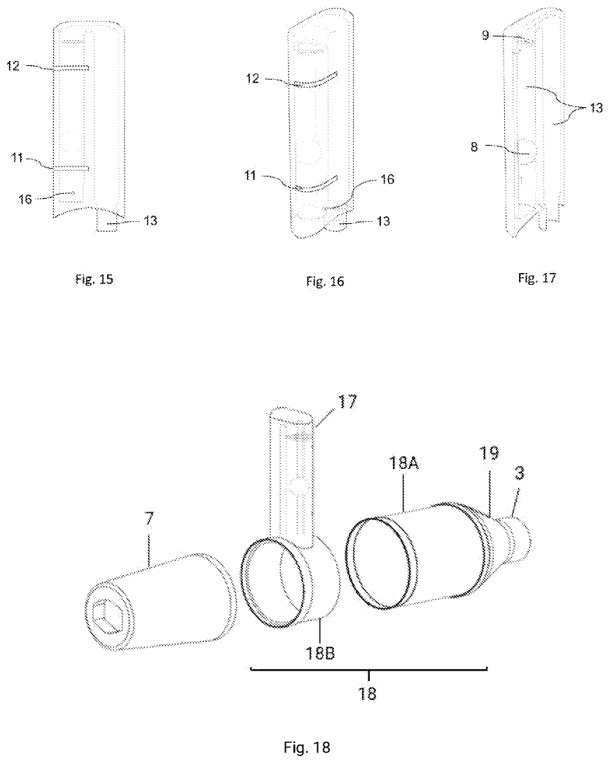

As can be seen in FIGS. 15, 16 and 17, the flow indicator (17) comprises a plurality of flow levels, a ball (8), a ball retainer (9), a flow passage conduit (13), a flow orifice (16) and a housing 17a and 17b. These parts are removable to facilitate the washing of the device.

In a preferred embodiment of the invention, the flow indicator has a flow level of 30 cm H₂O (11) and a flow level of 60 cm H₂O (12), since it has been established that for optimal delivery of the drug, the ideal flow of an inspiration through the inhalation chamber must be between 30 L/min and 60 L/min, so that the medicament manages to penetrate to the most distal areas of the lung.

In one embodiment, the inhalation flow chamber (1), allows coupling to the outer diameter of the mixed connector (3) the oronasal mask (2), as can be seen in FIGS. 4, 6 and 12, or the mouth of the patient, and by the inner diameter of the mixed connector (3), the tracheostomy cannula, as can be seen in FIG. 2.

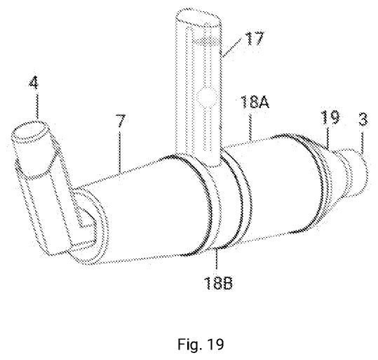

In another embodiment, the cylinder (18) comprises two disassembled sections (18A, 18b), wherein the flow indicator (17) is in connection with the second cylinder section (18b), through the connection of the flow passage conduit (13) with the coupling port (20), as shown in FIGS. 18 and 19. In this embodiment the lid for the chamber (7) has a longer length.

To achieve proper operation of the inhalation flow chamber, the metered-dose inhaler (4) must first be shaken according to institutional protocols, to immediately attach it to the docking port of the metered-dose inhaler (6).

In pediatric patients or patients with impaired consciousness, the oronasal mask (2) must first be attached to the mixed connector (3), and then the patient's face must be placed against the oronasal mask (2), verifying the elevation of the ball (8), then activating the metered-dose inhaler (4) following the institutional inhalation protocols.

In patients with loss of facial seal due to facial dimorphism, compromised skin integrity such as burns or abundant beard, presence of nasogastric tubes or reliance on oxygen therapy systems such as nasal cannulas, or tracheostomy, the patient should be asked to breath out, then adjusting the mixed connector (3) to the lips or tracheostomy.

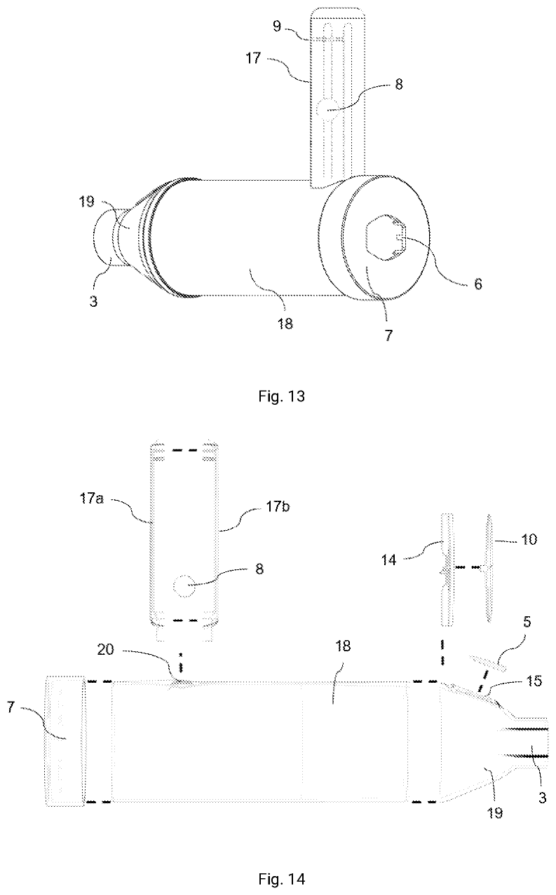

Subsequently, the patient is asked to start the inspiration and by effect of the suction generated, the air enters through the flow orifice (16) that crosses the cover of the flow indicator (17), passing through the passageway for the flow passage (13), which elevates the ball (8). The patient is asked to constantly hold the ball (8) elevated between the guides of the flow levels of 30 cm H₂O (11) and 60 cm H₂O (12), and at that time the activation of the metered dose inhaler (4) is performed. In this way the drug is suspended inside the chamber, passing through the inhalation unidirectional membrane (10) and reaching the distal airway. If the inspiratory flow is very high, the ball (8) will be retained by the ball retainer (9), see FIG. 13.

Upon exhalation, the air exits through the exhalation window (15), elevating the exhalation unidirectional membrane (5), sealing the spacer chamber by containing the inhalation unidirectional membrane (10) against the valve seal (14), preventing the loss of medication contained inside the chamber. This process is repeated according to medical prescription.

1. An inhalation flow chamber (1) comprising:
a mixed connector (3), an anterior body (19) with an exhalation window (15) housing an exhalation unidirectional membrane (5), arranged towards the proximal section of the inhalation chamber;
an inhalation unidirectional membrane (10), a valve seal (14), a cylinder (18), a coupling port for the pressure indicator (20) and a flow indicator (17), arranged in the middle section of the inhalation chamber;
a chamber lid (7) and a coupling port for the metered dose inhaler (6), arranged in the distal section of the inhalation chamber;
wherein the mixed connector (3) is coupled to the anterior body (19), which is coupled to the proximal end of the cylinder (18), within which the unidirectional inhalation membrane (10) and the valve seal (14), which supports said membrane are arranged;
wherein the cylinder (18) is in connection with the chamber lid (7) at its distal end;
wherein the flow indicator (17) is in connection with the cylinder (18), through the connection of a flow passage conduit (13) with the coupling port (20);
wherein the flow indicator (17) comprises a flow level of 30 L/min (11) and a flow level of 60 L/min (12);
wherein the cylinder (18) comprises two dismantlable sections (18A, 18b); and
wherein the flow indicator (17) is in connection with the second cylinder section (18b), through the connection of the flow passage conduit (13) with the coupling port (20).

2. The inhalation chamber according to claim 1, wherein the flow indicator (17) comprises a plurality of flow levels, a ball (8), a ball retainer (9), a flow passage conduit (13), a flow orifice (16), and a housing 17a and 17b.

3. The inhalation chamber according to claim 1, comprising an oronasal mask (2) coupled to the mixed connector (3).

4. The inhalation chamber according to claim 1, further comprising a metered dose inhaler (4).
